SEQUENCE LISTING (13 sequences)

SEQ ID NO: 1 - DNA, 723 bp, synthetic construct
  source 1..723: mol_type = other DNA
atggcccgccgccgccatcgcggcccccgccgcccccggccgcccgggcccacgggcgcggtcccaaccgcacagtcccaggtaacctccacgcccaactcggaacccgtggtcaggagcgcgcccgcggccgccccgccgccgccccccgccagtgggcccccgccttcttgttcgctgctgctgcgccagtggctccacgttcccgagtccgcgtccgacgacgacgacgacgactggccggacagccccccgcccgagccggcgccagaggcccggcccaccgccgccgccccccgcccccggtccccaccgcccggcgcgggcccggggggcggggctaacccctcccaccccccctcacgccccttccgccttccgccgcgcctcgccctccgcctgcgcgtcaccgcagagcacctggcgcgcctgcgcctgcgacgcgcgggcggggagggggcgccgaagccccccgcgacccccgcgacccccgcgacccccacgcgggtgcgcttctcgccccacgtccgggtgcgccacctggtggtctgggcctcggccgcccgcctggcgcgccgcggctcgtgggcccgcgagcgggccgaccgggctcggttccggcgccgggtggcggaggccgaggcggtcatcgggccgtgcctggggcccgaggcccgtgcccgggccctggcccgcggagccggcccggcgaactcggtctaa

SEQ ID NO: 2 - DNA, 366 bp, synthetic construct
  source 1..366: mol_type = other DNA
atgcaagtgcagctggtgcaaagcggcgtggaagtgaagaaacctggcgccagcgtgaaagtgtcctgcaaggcctctggatatacatttaccaattactacatgtactgggtgcggcaggccccaggccagggcctggaatggatgggcggcatcaaccctagcaacggcggtacaaacttcaacgagaagttcaagaacagagtgaccctgaccaccgacagcagcaccacaaccgcctacatggaactgaagagcctgcagttcgacgacaccgccgtgtactactgcgccaggcgggactacagattcgacatgggctttgattactggggacagggcacaacagtcaccgtgtccagcggc

SEQ ID NO: 3 - DNA, 348 bp, synthetic construct
  source 1..348: mol_type = other DNA
gagatcgtgctgacccagagccctgctacactctccctgagccccggcgagagagccaccctgagctgcagagccagcaagggcgtttctacatctggctacagctacctgcactggtaccagcagaagcccggccaggctcctagactgctgatctacctggcctcttacctggagagcggcgtccccgctagattcagcggctctggcagcggaaccgatttcaccctgaccatcagctcccttgagcctgaggacttcgccgtgtactattgtcagcacagcagagatctgcctctgacattcggcggaggcaccaaggtggaaatcaagcggaccgtggcctag

SEQ ID NO: 4 - DNA, 42 bp, synthetic construct
  source 1..42: mol_type = other DNA
ggcggaggctctggcggcggaggaagcggaggcggcggcagc

SEQ ID NO: 5 - DNA, 987 bp, synthetic construct
  source 1..987: mol_type = other DNA
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttag

SEQ ID NO: 6 - DNA, 762 bp, synthetic construct
  source 1..762: mol_type = other DNA
atgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 7 - DNA, 580 bp, synthetic construct
  source 1..580: mol_type = other DNA
tccgcccctctccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaacc

SEQ ID NO: 8 - DNA, 24 bp, synthetic construct
  source 1..24: mol_type = other DNA
gttgcctgtcaaactctaccaccc

SEQ ID NO: 9 - DNA, 20 bp, synthetic construct
  source 1..20: mol_type = other DNA
caatgcaaaagcctttgacg

SEQ ID NO: 10 - DNA, 24 bp, synthetic construct
  source 1..24: mol_type = other DNA
caccgaataacacataaatttggc

SEQ ID NO: 11 - DNA, 20 bp, synthetic construct
  source 1..20: mol_type = other DNA
ggcgagggcgatgccaccta

SEQ ID NO: 12 - DNA, 2335 bp, synthetic construct
  source 1..2335: mol_type = other DNA
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttagctcgactccgcccctctccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 13 - DNA, 756 bp, synthetic construct
  source 1..756: mol_type = other DNA
atgcaagtgcagctggtgcaaagcggcgtggaagtgaagaaacctggcgccagcgtgaaagtgtcctgcaaggcctctggatatacatttaccaattactacatgtactgggtgcggcaggccccaggccagggcctggaatggatgggcggcatcaaccctagcaacggcggtacaaacttcaacgagaagttcaagaacagagtgaccctgaccaccgacagcagcaccacaaccgcctacatggaactgaagagcctgcagttcgacgacaccgccgtgtactactgcgccaggcgggactacagattcgacatgggctttgattactggggacagggcacaacagtcaccgtgtccagcggcggcggaggctctggcggcggaggaagcggaggcggcggcagcgagatcgtgctgacccagagccctgctacactctccctgagccccggcgagagagccaccctgagctgcagagccagcaagggcgtttctacatctggctacagctacctgcactggtaccagcagaagcccggccaggctcctagactgctgatctacctggcctcttacctggagagcggcgtccccgctagattcagcggctctggcagcggaaccgatttcaccctgaccatcagctcccttgagcctgaggacttcgccgtgtactattgtcagcacagcagagatctgcctctgacattcggcggaggcaccaaggtggaaatcaagcggaccgtggcctag

(19) United States
(12) Patent Application Publication
(10) Pub. No.: US 2025/0352619 A1
(43) Pub. Date: Nov. 20, 2025

(54) RECOMBINANT HSV-1 VECTOR FOR ENCODING IMMUNOSTIMULATORY FACTOR AND ANTI-IMMUNE CHECKPOINT ANTIBODY

(71) Applicants: WUXI APPTEC (SHANGHAI) CO., LTD., Shanghai (CN); SHANGHAI ARK BIOPHARMACEUTICAL CO., LTD., Shanghai (CN)

(72) Inventors: Junmei ZHANG, Shanghai (CN); Wei MA, Shanghai (CN); Jim Zhen WU, Shanghai (CN); Gang ZOU, Shanghai (CN); Zhao GAO, Shanghai (CN)

(21) Appl. No.: 18/868,467

(22) PCT Filed: May 22, 2023
(86) PCT No.: PCT/CN2023/095606

§ 371 Date: Nov. 22, 2024

(30) Foreign Application Priority Data
May 23, 2022 (CN) 202210618608.5

(51) Int. Cl. A61K 38/20; A61K 39/00; A61K 39/395; A61K 48/00

(52) U.S. Cl. CPC A61K 38/208; A61K 39/39558; A61K 48/0058; A61K 2039/53; A61K 2039/585; C07K 2317/622; C12N 2710/16632

(57) ABSTRACT

The present disclosure provides a modified HSV-1 vector. The HSV-1 vector comprises an exogenous nucleotide sequence encoding an immunostimulatory factor and/or an anti-immune checkpoint antibody. The HSV-1 vector of the present disclosure can be used for treating cancers.

### TECHNICAL FIELD

The present disclosure relates to the fields of immunology and gene delivery. More specifically, the present application relates to a recombinant HSV-1 vector carrying and expressing immunostimulatory factor and anti-immune checkpoint antibody.

### BACKGROUND

Oncolytic virus is a virus that has been genetically modified or naturally occurring. They preferentially replicate in cancer cells and lyse cancer cells, and induce the body to produce anti-tumor immune responses, but have little impact on normal cells, making them a widely applicable anti-tumor treatment method. At present, various pathogens including herpes simplex virus, adenovirus, poxvirus, and coxsackievirus have been used in the preparation of oncolytic viruses.

Among them, herpes simplex virus has attracted much attention in the preparation of oncolytic viruses, as it has the following advantages as an oncolytic virus vector: 1. clear genome and viral gene product functions; 2. large genome that is available to accommodate multiple exogenous genes; 3. no risk of viral genome integration; 4. mature modification technology; 5. verified safety after administration to the human body; 6. strong immunogenicity.

At present, oncolytic viruses can be divided into more than ten types according to virus types. Among them, oncolytic herpes simplex virus type 1 (HSV-1) has become the first choice for genetic engineering tumor therapeutic medicaments at home and abroad due to the advantages of large gene capacity, short replication cycle, high infection efficiency, and the ability to insert multiple therapeutic genes in the viral vector used. However, due to the different genetic modifications, oncolytic effects, and safety of different oncolytic viruses, their applicable tumor indications and therapeutic effects may also vary (Eager R M, Nemunaitis J. Clinical development directions in oncolytic virotherapy [J]. Cancer Gene Ther, 2011, 18(5):305-317). The existing oncolytic viruses all have drawbacks such as high toxicity and strong side effects, poor safety, and significantly limited therapeutic doses of oncolytic viruses, which pose serious challenges to tumor treatment research (Liu T C, Galanis E, Kirn D. Clinical trial results with oncolytic virus therapy: a century of promise, a decade of progress [J]. Nat Clin Pract Oncol, 2007, 4 (2): 101-117). Due to the low specificity and safety of oncolytic viruses, the dosage will be reduced to avoid serious side effects on the body. This will affect the clinical treatment efficacy of oncolytic viruses and lead to certain safety hazards.

At present, the anti-tumor immune effect induced by oncolytic herpes simplex virus (oHSV) can be enhanced by assembling anti-tumor immunoregulation factors. At the same time, anti-tumor immunoregulation factors can reverse the immunosuppressive effect of the tumor microenvironment, promote inflammatory cell infiltration, and enhance the anti-tumor effect of oncolytic virus.

Therefore, it is still necessary to develop new oncolytic viruses to achieve low toxicity and high effectiveness of oncolytic virus therapy, and further enhance anti-tumor effects by combining the expression of anti-tumor immunoregulation factors.

### SUMMARY

The present disclosure provides a recombinant herpes simplex virus type 1 (hereinafter also referred to as HSV-1, HSV-1 virus or HSV-1 vector) comprising a modified HSV-1 genome, wherein the modification comprises a deletion of the ICP34.5 gene in a HSV1-KOS strain genome. Preferably, the modification results in the deletion of two copies of the ICP34.5 gene.

In one aspect, the present disclosure provides a recombinant HSV-1 vector comprising a modified HSV-1 genome, wherein the modification comprises a deletion of the ICP34.5 gene in the HSV1-KOS strain genome, the vector comprises: (a) a first exogenous nucleic acid sequence encoding an immunostimulatory factor; (b) a second exogenous nucleic acid sequence encoding an anti-immune checkpoint antibody; wherein the first exogenous nucleic acid sequence or the second exogenous nucleic acid sequence is inserted between the UL26 and UL27 genes of the HSV1-KOS strain genome, and the insertion of the first exogenous nucleic acid sequence does not interfere with an expression of HSV-1. Preferably, the second exogenous nucleic acid sequence is inserted between the UL26 and UL27 genes of the HSV1-KOS strain genome, and the insertion of the second exogenous nucleic acid sequence does not interfere with the expression of HSV-1.

In some embodiments, the recombinant HSV-1 vector as described above, wherein the first exogenous nucleic acid sequence encoding the immunostimulatory factor is inserted into a region of the deleted ICP34.5 gene in the HSV1-KOS strain genome or between the UL3 and UL4 genes of the HSV1-KOS strain genome, and the second exogenous nucleic acid sequence encoding the anti-immune checkpoint antibody is inserted between the UL26 and UL27 genes of the HSV1-KOS strain genome.

In some embodiments, the recombinant HSV-1 vector as described above, wherein the first exogenous nucleic acid sequence encoding the immunostimulatory factor is inserted between the UL26 and UL27 genes of the HSV1-KOS strain genome, and the second exogenous nucleic acid sequence encoding the anti-immune checkpoint antibody is inserted in a region of the deleted ICP34.5 gene in the HSV1-KOS strain genome or between the UL3 and UL4 genes of the HSV1-KOS strain genome.

In some embodiments, the recombinant HSV-1 vector as described above, wherein the immunostimulatory factor is at least one selected from GM-CSF, IL-2, IL-12, IL-15, IL-24, and IL-27; and the immune checkpoint is at least one selected from PD-1, CTLA-4, VISTA, LAG-3, TIGIT, and PD-L1.

In some embodiments, the recombinant HSV-1 vector as described above, wherein the vector comprises: (a) a first exogenous nucleic acid sequence encoding IL-12; (b) a second exogenous nucleic acid sequence encoding an anti-PD-1 antibody; wherein the second exogenous nucleic acid sequence of the anti-PD-1 antibody is inserted between the UL26 and UL27 genes of the HSV1-KOS strain genome.

In some embodiments, the recombinant HSV-1 vector as described above, wherein the first exogenous nucleic acid sequence encoding IL-12 is inserted into a region of the deleted ICP34.5 gene in the HSV1-KOS strain genome or between the UL3 and UL4 genes of the HSV1-KOS strain genome.

In some embodiments, the recombinant HSV-1 vector as described above, wherein the deletion of the ICP34.5 gene is a two-copy deletion of the ICP34.5 gene or a deletion of amino acid positions 1-146 of the N-terminal sequence of the ICP34.5 gene.

In some embodiments, the recombinant HSV-1 vector as described above, wherein the nucleic acid sequence of the ICP34.5 gene comprises the sequence shown in SEQ ID NO: 1.

In some embodiments, the recombinant HSV-1 vector as described above, wherein the immunostimulatory factor such as IL-12 or anti-immune checkpoint antibody such as anti-PD-1 antibody is from human or murine.

In some embodiments, the recombinant HSV-1 vector as described above, wherein the anti-immune checkpoint antibody such as anti-PD-1 antibody is an intact antibody, a single chain antibody (scFv), or an antibody fragment.

In some embodiments, the recombinant HSV-1 vector as described above, wherein the nucleic acid sequence of the anti-PD-1 antibody comprises the sequence shown in SEQ ID NO: 2.

In some embodiments, the recombinant HSV-1 vector as described above, wherein the first exogenous nucleic acid sequence encoding IL-12 comprises a sequence obtained by tandemly linking SEQ ID NO: 3 and SEQ ID NO: 4 via an IRES, preferably, the IRES sequence comprises a sequence shown in SEQ ID NO: 5.

In some embodiments, the recombinant HSV-1 vector as described above, wherein the vector further comprises a promoter sequence operably linked to the first exogenous nucleic acid sequence or the second exogenous nucleic acid sequence.

In some embodiments, the recombinant HSV-1 vector as described above, wherein the promoter is at least one selected from CMV, SV40, EF1A, CBh, and CAG promoters.

In some embodiments, the recombinant HSV-1 vector as described above, wherein the deletion of the ICP34.5 gene is a two-copy gene deletion, the insertion of the first exogenous nucleic acid sequence encoding the immunostimulatory factor such as IL-12 is a two-copy insertion or a single-copy insertion, and the insertion of the second exogenous nucleic acid sequence encoding the anti-immune checkpoint antibody such as anti-PD-1 antibodies is a single copy insertion. In an optional embodiment, the insertion of the first exogenous nucleic acid sequence encoding the immunostimulatory factor is a two-copy insertion, and the first exogenous nucleic acid sequences inserted at each insertion site are the same. In an optional embodiment, the insertion of the first exogenous nucleic acid sequence encoding the immunostimulatory factor is a two-copy insertion, and the first exogenous nucleic acid sequences inserted at each insertion site are different.

Another aspect hereof relates to a pharmaceutical composition or kit comprising a therapeutically effective amount of the recombinant HSV-1 vector as previously described, and one or more pharmaceutically acceptable carriers, diluents, buffers, or excipients.

Another aspect hereof relates to the use of the recombinant HSV-1 vector or the pharmaceutical composition or kit as previously described in the preparation of a medicament for treating and/or preventing a cancer.

In some embodiments, the cancer is at least one selected from a gallbladder cancer, bladder cancer, basal cell tumor, extrahepatic cholangiocarcinoma, colorectal cancer, endometrial cancer, cervical cancer, esophageal cancer, breast cancer, Ewing's sarcoma, prostate cancer, gastric cancer, glioma, Hodgkin's lymphoma, laryngeal cancer, liver cancer, lung cancer, melanoma, mesothelioma, pancreatic cancer, renal cancer, peripheral nerve tumor, skin and plexiform neurofibroma, leiomyomatoid tumor, fibroma, uterine fibroids, leiomyosarcoma, thyroid cancer, ascites, mesothelioma, salivary gland tumor, mucoepidermoid carcinoma of the salivary gland, acinar cell carcinoma of the salivary gland, gastrointestinal stromal tumor (GIST), tumors causing accumulation of fluid in potential spaces of the body, pleural effusion, pericardial effusion, peritoneal effusion, giant cell tumor, pigmented villonodular synovitis (PVNS), tenosynovial giant cell tumor (TGCT), and sarcoma, wherein the liver cancer is preferably hepatocellular carcinoma (HCC), cholangiocellular carcinoma or mixed type of liver cancer; the lung cancer is preferably non-small cell cancer or small cell lung cancer; the peripheral nerve tumor is preferably a malignant peripheral nerve sheath tumor (MPNST); the thyroid cancer is preferably at least one selected from papillary thyroid cancer, anaplastic thyroid cancer, medullary thyroid cancer, follicular thyroid cancer, and Hurthle cell carcinoma; the ascites is preferably malignant ascites; the giant cell tumor is a giant cell tumor of bone or a giant cell tumor of the tendon sheath; the salivary gland tumor is preferably mucoepidermoid carcinoma of the salivary gland or acinar cell carcinoma of the salivary gland; the laryngeal cancer is preferably laryngeal mucoepidermoid carcinoma; the colorectal cancer is preferably colon cancer or rectal cancer.

In some embodiments, the administration of the medicament is local administration or systemic administration, wherein the local administration includes intratumoral injection, the systemic administration includes oral administration and intravascular injection. Wherein, the intravascular injection is preferably intravenous injection

### Beneficial Effects of the Invention

The oncolytic virus of the present disclosure has high expression levels, good stability, and high oncolytic activity. By modifying the virus structure, such as the deletion of the ICP34.5 gene in the HSV1-KOS strain genome, and the creative introduction of immune stimulator and/or immune checkpoint antibody such as PD-1 antibody at the insertion site of the UL26/27 genes, the obtained modified recombinant herpes simplex virus type 1 has better tumor suppressive effect compared to those without modification.

### BRIEF DESCRIPTION OF THE DRAWINGS

FIG. 1 shows a schematic diagram of a portion of the structures in the genome of the original virus GHSV-UL46 (ATCC-VR-1544, GHSV-UL46).

FIG. 2 shows a schematic diagram of a portion of the structure in the genome of the recombinant herpes simplex virus vector HSV1-hIL12(34.5 KO)-αhPD1(UL26/27) that expresses an immunostimulatory factor.

FIG. 3 shows the expression of the immunostimulatory factor IL12 in cells.

FIG. 4 shows the growth curves of different viral constructs on African green monkey kidney cell Vero.

FIG. 5 shows the growth curves of different constructs of the same viral vector with different insertion sites on African green monkey kidney cell Vero.

FIG. 6 shows the results of plaque assay at 48 hours after viral infection.

FIG. 7 shows the killing effect of recombinant herpes simplex virus vector HSV1-hIL12(34.5 KO) expressing immunostimulatory factor IL12 and anti-hPD1 antibody on glioma cell U87-MG, and laryngeal epidermoid carcinoma cell Hep-2.

FIG. 8 shows the killing effect of recombinant herpes simplex virus vector HSV1-hIL12(34.5 KO) expressing immunostimulatory factor IL12 and anti-hPD1 antibody on glioma cell LN229, and melanoma cell A375.

FIG. 9 shows the killing effect of recombinant herpes simplex virus vector HSV1-hIL12(34.5 KO) expressing immunostimulatory factor IL12 and anti-hPD1 antibody on melanoma cell SK-Mel-28, and bladder cancer cell 5637.

FIG. 10 shows the killing effect of recombinant herpes simplex virus vector HSV1-hIL12(34.5 KO) expressing immunostimulatory factor IL12 and anti-hPD1 antibody on non-small cell lung cancer cell A549, and hepatocellular carcinoma cell HepG2.

FIG. 11 shows the killing effect of recombinant herpes simplex virus vector HSV1-hIL12(34.5 KO) expressing immunostimulatory factor IL12 and anti-hPD1 antibody on prostate cancer cell LN-Cap, colorectal cancer cell HCT116, and breast cancer MCF-7.

FIG. 12 shows the killing effect of HSV1-hIL12(34.5 KO)-αhPD1(UL26/27) and other vector (HSV1-hIL12(34.5 KO)-αhPD1(UL3/4)) on non-small cell lung cancer cell A549, laryngeal epidermoid carcinoma cell Hep-2, and melanoma cell SK-Mel-28.

FIG. 13 shows the killing effect of HSV1-hIL12(34.5 KO)-αhPD1(UL26/27) and other vector (HSV1-hIL12(34.5 KO)-αhPD1(UL3/4)) on colorectal cancer cells HCT116, glioma cells LN229, and melanoma cells A375.

FIG. 14 shows the in vivo oncolytic activity of HSV1-hIL12(34.5 KO)-αhPD1(UL26/27).

### DETAILED DESCRIPTION

The present disclosure is described in detail below according to embodiments and in conjunction with the accompanying drawings. The above aspects of the present disclosure and other aspects of the present disclosure will be apparent in the following detailed description. The scope of the present disclosure is not limited to the following examples.

The antibodies in the present disclosure are multispecific, and can be humanized, single-chain, chimeric, synthetic, recombinant, heterozygous, mutant, and grafted andtibodies; the antibody format in the present disclosure is a scFv spliced by antibody light chain variable regions (VL) and antibody heavy chain variable regions (VH). The antibody light chain variable region (VL) and antibody heavy chain variable region (VH) can be further subdivided into hypervariable regions called complementarity determining regions (CDR), and interspersed more conserved regions called framework regions (FWR). The CDRs of the antibodies and antigen-binding fragments disclosed herein are defined or identified by Kabat numbering. In one embodiment, each VH and VL generally includes 3 CDRs and 4 FWRs arranged in the following order from the amino end to the carboxy end: FWR1, CDR1, FWR2, CDR2, FWR3, CDR3, FWR4. The CDRs of the antibodies and antigen-binding fragments disclosed herein are defined or recognized by Kabat numbering.

As used herein, the term “antibody fragment” or “antigen-binding fragment” is a portion of an antibody, e.g., F(ab′)₂, F(ab′)₂, Fab′, Fab, Fv, scFv, and the like. Regardless of structure, the antibody fragment binds to the same antigen recognized by the complete antibody. The term “antibody fragment” includes aptamers, mirror-image isomers and diabodies. The term “antibody fragment” also includes any synthetic or genetically engineered protein that acts like an antibody by binding to a specific antigen to form a complex.

As used herein, “cancer” or “tumor” as used interchangeably herein refers to a group of diseases that can be treated in accordance with the present disclosure and involve abnormal or uncontrolled cell growth which may invade or spread to other parts of the body. Not all tumors are cancerous; benign tumors do not spread to other parts of the body. Possible physical signs and symptoms include new lumps, abnormal bleeding, prolonged coughing, unexplained weight loss and changes in bowel movements. There are over 100 different known cancers affecting humans. The present disclosure is preferably applicable to solid tumors. Non-limiting examples of tumors or cancers include gallbladder cancer, bladder cancer, basal cell tumor, extrahepatic cholangiocarcinoma, colorectal cancer, endometrial cancer, cervical cancer, esophageal cancer, breast cancer, Ewing's sarcoma, prostate cancer, gastric cancer, glioma, Hodgkin's lymphoma, laryngeal cancer, liver cancer, lung cancer, melanoma, mesothelioma, pancreatic cancer, renal cancer, peripheral nerve tumor, skin and plexiform neurofibroma, leiomyomatoid tumor, fibroma, uterine fibroids, leiomyosarcoma, thyroid cancer, ascites, mesothelioma, salivary gland tumor, mucoepidermoid carcinoma of the salivary gland, acinar cell carcinoma of the salivary gland, gastrointestinal stromal tumor (GIST), tumors causing accumulation of fluid in potential spaces of the body, pleural effusion, pericardial effusion, peritoneal effusion, giant cell tumor, pigmented villonodular synovitis (PVNS), tenosynovial giant cell tumor (TGCT), and sarcoma. Wherein, the liver cancer may be hepatocellular carcinoma (HCC), cholangiocellular carcinoma or mixed type of liver cancer; the lung cancer may be non-small cell cancer or small cell lung cancer; the peripheral nerve tumor may be a malignant peripheral nerve sheath tumor (MPNST); the thyroid cancer is at least one selected from papillary thyroid cancer, anaplastic thyroid cancer, medullary thyroid cancer, follicular thyroid cancer, and Hurthle cell carcinoma; the ascites may be malignant ascites; the giant cell tumor is a giant cell tumor of bone or a giant cell tumor of the tendon sheath; the salivary gland tumor may be mucoepidermoid carcinoma of the salivary gland or acinar cell carcinoma of the salivary gland; the laryngeal cancer is preferably laryngeal mucoepidermoid carcinoma; the colorectal cancer is preferably colon cancer or rectal cancer.

As used herein, the term “treatment” refers to therapeutic treatments and prophylactic measures for the purpose of preventing or slowing down (mitigating) undesired physiological changes or disorders, such as the progression of cancer. Beneficial or desired clinical outcomes include, but are not limited to, alleviating of symptoms, reducing the extent of the disease, stabilizing (i.e., no worsening) the disease state, delaying or slowing down the disease progression, improving or alleviating the disease state, and disappearance of the symptoms (whether partial or total), whether detectable or undetectable. “Treatment” also means prolonged survival compared to what would have been expected without treatment. Patients in need of treatment include those who already have a disease or condition, as well as those who are susceptible to a disease or condition, or those who are preventing a disease or condition.

“Subject” means any subject, particularly a mammalian subject, for whom diagnosis, prognosis or treatment is desired. Mammalian subjects include human or non-human animals such as dogs, cats, guinea pigs, rabbits, rats, mice, horses, cows, dairy cows and the like.

The term “administration” as used herein refers to the introduction of a medicament or pharmaceutical composition into a subject.

The term “effective amount” refers to the smallest amount of a medicament or pharmaceutical composition required to produce a specific physiological effect.

The term “vector” refers to a nucleic acid molecule capable of transferring or transporting another nucleic acid molecule. In some embodiments, the vectors used in the present disclosure are viral vectors of herpes simplex type 1 virus.

### Recombinant HSV-1 Vectors

In one aspect, the present disclosure provides a recombinant HSV-1 vector comprising a modified HSV-1 genome, wherein the modification comprises a deletion of the ICP34.5 gene in the HSV1-KOS strain genome, the vector comprising (a) a first exogenous nucleic acid sequence encoding an immunostimulatory factor such as IL-12; and (b) a second exogenous nucleic acid sequence encoding an anti-immune checkpoint such as a PD-1 antibody; wherein the second exogenous nucleic acid sequence encoding the anti-immune checkpoint such as an antibody to PD-1 is inserted between the UL26 and UL27 genes of the HSV1-KOS strain genome, and wherein the insertion of the first exogenous nucleic acid sequence or the second exogenous nucleic acid sequence does not interfere with the expression of HSV-1. Wherein, the first exogenous nucleic acid sequence encoding the immunostimulatory factor such as IL-12 is inserted in a region of the deleted ICP34.5 gene in the HSV1-KOS strain genome or between the UL3 and UL4 genes of the HSV1-KOS strain genome. Wherein, the deletion of the ICP34.5 gene is a deletion of two-copy of the ICP34.5 gene or a deletion of amino acid positions 1-146 of the N-terminal sequence of the ICP34.5 gene. The nucleic acid sequence of the ICP34.5 gene comprises or is SEQ ID NO: 1. It should be understood by those skilled in the art that the immunostimulatory factor may be preferably at least one selected from GM-CSF, IL-2, IL-12, IL-15, IL-24 and IL-27. The immune checkpoint may be preferably at least one selected from PD-1, CTLA-4, VISTA, LAG-3, TIGIT and PD-L1. The vector further comprises a promoter sequence or other element operably linked to the first exogenous nucleic acid sequence or the second exogenous nucleic acid sequence. Preferably, wherein the deletion of the ICP34.5 gene is a deletion of a two-copy gene, wherein the insertion of the first exogenous nucleic acid sequence encoding the immunostimulatory factor is a two-copy or a single-copy insertion, and the insertion of the second exogenous nucleic acid sequence encoding the anti-immune checkpoint antibody is a single-copy insertion.

The HSV1-KOS strain is derived from ATCC-VR-1544, GHSV-UL46.

### Pharmaceutical Compositions

The oncolytic virus can be prepared in suitable pharmaceutically acceptable carriers or excipients. These preparations contain preservatives to prevent the growth of microorganisms under usual conditions of storage and use. Forms of the medicament suitable for injection include sterile aqueous solutions or dispersions, and sterile powders for the temporary preparation of sterile injectable solutions or dispersions. In all cases, the formulation must be sterile and must be fluid so as to be easily injectable. It must be stable under production and storage conditions and must be protected against contamination by microorganisms such as bacteria and fungi. As used herein, “pharmaceutically acceptable carrier” includes any and all solvents, dispersing media, carriers, coatings, diluents, antibacterial and antifungal agents, isotonic and absorption delaying agents, buffers, carrier solutions, suspensions, colloids and the like. Mediums and agents for use with pharmaceutical active substances are well known in the art. Except any conventional media or reagents that are incompatible with the active ingredient, it is expected that other media or reagents can be used in the therapeutic compositions. Supplemental active ingredients may also be incorporated into the compositions. “Pharmaceutically acceptable” refers to molecular entities and components that do not produce allergic or similar adverse reactions when administered to humans. The preparation of aqueous compositions containing proteins as active ingredients is well understood in the art. Typically, such compositions are prepared as injections, whether as liquid solutions or suspensions; solid forms suitable for dissolution or suspension in a liquid prior to injection may also be prepared.

### EXAMPLES

The present disclosure is further described below with reference to Examples, but these examples do not limit the scope of the present disclosure. Experimental methods without specific conditions indicated in the examples of the present disclosure, generally follow conventional conditions. Reagents without specific sources indicated, are conventional reagents purchased in the market.

The experimental materials used in the following Examples are shown in TABLES 1 and 2.

TABLE 1
Key Reagents

| Reagent | Brand - Catalog No. |
|---|---|
| 2* TransStart FastPfu PCR SuperMix | Transgene-AS221 |
| Bulk agarose gel DNA recovery kit | TIANGEN-DP210-02 |
| 2x NEB builder | NEB-M5520A |
| T-Fast competent cell | TIANGEN-CB109 |
| 2x phanta master mix | Vazyme-p511-aa |
| Q5 ® High-Fidelity DNA Polymerase | NEB-M0491 |
| LB medium | Invitrogen-12780052 |
| Ampicillin | Tiangen-RT501 |
| EndoFree ® Plasmid Maxi Kit | QIAGEN-12362 |
| HindIII-HF | NEB-R3104V |
| XhoI | NEB-R0146V |
| XbaI | NEB-R0145S |
| SpeI | NEB-R0133S |
| DMEM | Gibco-11965-092 |
| Fetal bovine serum | ExCell Bio- FND500 |
| RPMI-1640 | Gibco-22400-089 |
| Cell Counting Kit (CCK8) | Liji-AC11L057 |
| Penicillin-Streptomycin Solution | Hyclone-SV30010 |
| 0.25% Trypsin-EDTA | Gibco-25200-072 |
| Mouse IL-12 p70 Quantikine ELISA Kit | R&D-M1270 |
| Human IL-12 p70 Quantikine HS ELISA Kit | R&D-HS120 |
| Lipofectamine 3000 Transfection Kit | invitrogen-L3000-015 |

TABLE 2
Key instruments

| Instruments | Brand | Model |
|---|---|---|
| PCR Amplifier | BIO-RAD | C1000TM Thermal Cycler |
| Electrophoresis Meter | BIO-RAD | PowerPac Basic |
| Gel Imaging System | BIO-RAD | ChemiDocTM MP Imaging |
| | | System |
| Centrifuge | Eppendorf | 5424R |
| Water Bath Cauldron | JULOBO | TW8 |
| Spectrophotometer | Thermo Fisher | Nanodrop 1000 |
| CO₂ Incubator | Thermo | HeraceII240i |
| microplate reader | Molecular devices | SpectraMaxM2e |

### Example 1: Genome Structure of Recombinant Herpes Simplex Virus Vector HSV1-hIL12(34.5 KO) -αhPD1(UL26/27) Expressing Immunostimulatory Factor

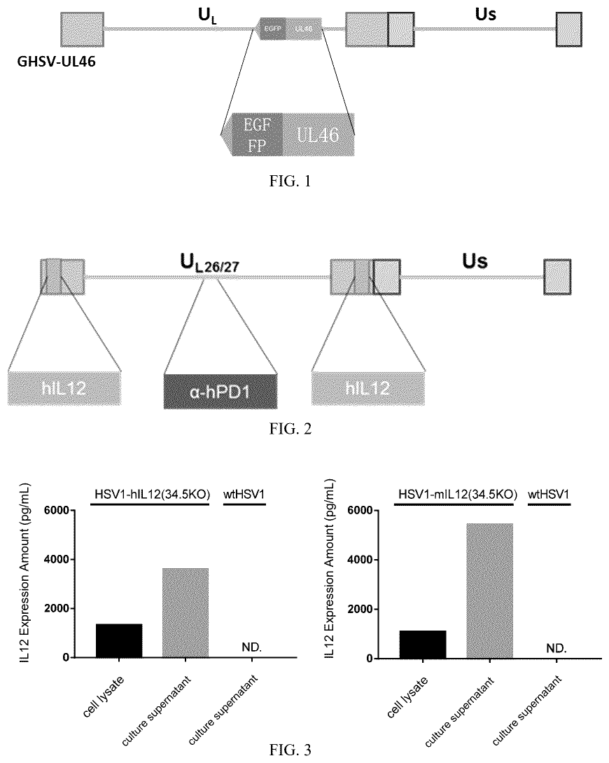

As shown in FIG. 2, in HSV1-hIL12(34.5 KO)-αhPD1(UL26/27) virus, the neurovirulence protein ICP34.5 (SEQ ID NO: 1) was replaced by hIL12 and the hPD-1 single chain antibody gene (SEQ ID NO:2) was inserted between the viral genomes UL26/27, wherein hIL12 consists of tandemly linked IL12B (SEQ ID NO: 5) and IL12A (SEQ ID NO: 6) via an internal ribosome entry site sequence (IRES, SEQ ID NO: 7).

The construction of the recombinant virus was achieved by accessible means of Red recombination based on conventional Bacterial Artificial Chromosome (BAC), gene editing technology such as CRISPR/Cas9 or the like. The specific construction method comprises the following steps: based on a laboratory passaged virus HSV1-KOS strain (ATCC-VR-1544, GHSV-UL46, a schematic diagram shown in FIG. 1), a neurovirulence protein ICP34.5 (SEQ ID NO: 1) was knocked out by a gene editing technology CRISPR/Cas9, and the ICP34.5 gene was deleted and replaced by an IL12 gene by a two-step method. Firstly, GFP gene was used for replacing ICP34.5 gene, and fluorescent cytopathy was selected; then, the GFP gene was replaced with IL12, and non-fluorescent cytopathy was selected. The two-step method greatly improved the work efficiency. The specific operations were as follows:
1) A donor plasmid containing the GFP gene was constructed, and a sgRNA plasmid (SEQ ID NO: 8) targeting ICP34.5 gene was simultaneously constructed. Vero cells were co-transfected by the GFP donor plasmid and the sgRNA plasmid, and were simultaneously infected by HSV1-KOS viruses, and cytopathy was subsequently observed. If ICP34.5 gene is successfully replaced by GFP gene, then green fluorescent cytopathy can be observed. The green fluorescent cytopathy was picked and HSV1 recombinant virus HSV1-ICP34.5 KO-GFP, in which ICP34.5 gene was successfully replaced by GFP gene, was purified by plaque experiment.
2) A donor plasmid containing an IL12 gene was constructed, and an sgRNA plasmid (SEQ ID NO: 11) targeting GFP gene was simultaneously constructed. Vero cells were co-transfected by the donor plasmid and the sgRNA plasmid, and were simultaneously infected by HSV1-ICP34.5 KO-GFP recombinant viruses. Cytopathy was subsequently observed. If the GFP gene is successfully replaced by the IL12 gene, cytopathy without green fluorescent can be observed. The cytopathy without green fluorescent was picked and the virus was purified by plaque experiment to obtain HSV1-hIL12(34.5 KO) virus; wherein hIL12 consists of tandemly linked IL12B (SEQ ID NO: 5) and IL12A (SEQ ID NO: 6) via an internal ribosome entry site sequence (IRES, SEQ ID NO: 7).
3) hPD-1 single-chain antibody gene was inserted between virus genomes UL26/27 (the gRNA sequence of UL26/27 is SEQ ID NO: 9) through a CRISPR/Cas9 two-step method to obtain an HSV1-hIL12(34.5 KO)-αhPD1(UL26/27) virus vector; wherein hPD1 consists of tandemly linked Heavy chain V (SEQ ID NO: 2) and Light chain V (SEQ ID NO: 3) via a linker (G4S)₃ (SEQ ID NO: 5).
4) hPD-1 single-chain antibody gene was inserted between virus genomes UL3/4 (the gRNA sequence of UL3/4 is SEQ ID NO: 10) through a CRISPR/Cas9 two-step method to obtain an HSV1-hIL12(34.5 KO)-αhPD1(UL3/4) virus vector; wherein hPD1 consists of tandemly linked Heavy chain V (SEQ ID NO: 2) and Light chain V (SEQ ID NO: 3) via a linker (G4S)₃ (SEQ ID NO: 5).

In addition, a mouse version recombinant herpes simplex virus vector HSV1-mIL12 (34.5 KO)-αmPD1 (UL26/27) inserted with a mouse-derived cytokine IL-12 and a mouse-derived PD-1 single-chain antibody gene was simultaneously constructed, and a target gene could be effectively expressed.

TABLE 3
Sequence Information

| Sequence | | |
|---|---|---|
| Information | Nucleic Acid Sequence | |
| ICP34.5 gene | atggcccgccgccgccatcgcggcccccgccgcccccggccgcccgggcccacgggcgcggtcccaa | SEQ ID |
| | ccgcacagtcccaggtaacctccacgcccaactcggaacccgtggtcaggagcgcgcccgcggccgcc | NO : 1 |
| | ccgccgccgccccccgccagtgggcccccgccttcttgttcgctgctgctgcgccagtggctccacgttcc | |
| | cgagtccgcgtccgacgacgacgacgacgactggccggacagccccccgcccgagccggcgccagag | |
| | gcccggcccaccgccgccgccccccgcccccggtccccaccgcccggcgcgggcccggggggcggg | |
| | gctaacccctcccaccccccctcacgccccttccgccttccgccgcgcctcgccctccgcctgcgcgtcac | |
| | cgcagagcacctggcgcgcctgcgcctgcgacgcgcgggcggggagggggcgccgaagccccccgc | |
| | gacccccgcgacccccgcgacccccacgcgggtgcgcttctcgccccacgtccgggtgcgccacctggt | |
| | ggtctgggcctcggccgcccgcctggcgcgccgcggctcgtgggcccgcgagcgggccgaccgggct | |
| | cggttccggcgccgggtggcggaggccgaggcggtcatcgggccgtgcctggggcccgaggcccgtg | |
| | cccgggccctggcccgcggagccggcccggcgaactcggtctaa | |
| Heavy chain V | atgcaagtgcagctggtgcaaagcggcgtggaagtgaagaaacctggcgccagcgtgaaagtgtcctgca | SEQ ID |
| | aggcctctggatatacatttaccaattactacatgtactgggtgcggcaggccccaggccagggcctggaat | NO : 2 |
| | ggatgggcggcatcaaccctagcaacggcggtacaaacttcaacgagaagttcaagaacagagtgaccct | |
| | gaccaccgacagcagcaccacaaccgcctacatggaactgaagagcctgcagttcgacgacaccgccgt | |
| | gtactactgcgccaggcgggactacagattcgacatgggctttgattactggggacagggcacaacagtca | |
| | ccgtgtccagcggc | |
| Light chain V | gagatcgtgctgacccagagccctgctacactctccctgagccccggcgagagagccaccctgagctgca | SEQ ID |
| | gagccagcaagggcgtttctacatctggctacagctacctgcactggtaccagcagaagcccggccaggct | NO : 3 |
| | cctagactgctgatctacctggcctcttacctggagagcggcgtccccgctagattcagcggctctggcagc | |
| | ggaaccgatttcaccctgaccatcagctcccttgagcctgaggacttcgccgtgtactattgtcagcacagca | |
| | gagatctgcctctgacattcggcggaggcaccaaggtggaaatcaagcggaccgtggcctag | |
| (G4S)₃ | ggcggaggctctggcggcggaggaagcggaggcggcggcagc | SEQ ID |
| | | NO : 4 |
| hIL12B | atgtgtcaccagcagttggtcatctcttggttttccctggtttttctggcatctcccctcgtggccatatgggaact | SEQ ID |
| | gaagaaagatgtttatgtcgtagaattggattggtatccggatgcccctggagaaatggtggtcctcacctgtg | NO : 5 |
| | acacccctgaagaagatggtatcacctggaccttggaccagagcagtgaggtcttaggctctggcaaaacc | |
| | ctgaccatccaagtcaaagagtttggagatgctggccagtacacctgtcacaaaggaggcgaggttctaag | |
| | ccattcgctcctgctgcttcacaaaaaggaagatggaatttggtccactgatattttaaaggaccagaaagaa | |
| | cccaaaaataagacctttctaagatgcgaggccaagaattattctggacgtttcacctgctggtggctgacga | |
| | caatcagtactgatttgacattcagtgtcaaaagcagcagaggctcttctgacccccaaggggtgacgtgcg | |
| | gagctgctacactctctgcagagagagtcagaggggacaacaaggagtatgagtactcagtggagtgcca | |
| | ggaggacagtgcctgcccagctgctgaggagagtctgcccattgaggtcatggtggatgccgttcacaagc | |
| | tcaagtatgaaaactacaccagcagcttcttcatcagggacatcatcaaacctgacccacccaagaacttgca | |
| | gctgaagccattaaagaattctcggcaggtggaggtcagctgggagtaccctgacacctggagtactccac | |
| | attcctacttctccctgacattctgcgttcaggtccagggcaagagcaagagagaaaagaaagatagagtctt | |
| | cacggacaagacctcagccacggtcatctgccgcaaaaatgccagcattagcgtgcgggcccaggaccg | |
| | ctactatagctcatcttggagcgaatgggcatctgtgccctgcagttag | |
| hIL12A | atgtggccccctgggtcagcctcccagccaccgccctcacctgccgcggccacaggtctgcatccagcgg | SEQ ID |
| | ctcgccctgtgtccctgcagtgccggctcagcatgtgtccagcgcgcagcctcctccttgtggctaccctggt | NO : 6 |
| | cctcctggaccacctcagtttggccagaaacctccccgtggccactccagacccaggaatgttcccatgcct | |
| | tcaccactcccaaaacctgctgagggccgtcagcaacatgctccagaaggccagacaaactctagaatttta | |
| | cccttgcacttctgaagagattgatcatgaagatatcacaaaagataaaaccagcacagtggaggcctgttta | |
| | ccattggaattaaccaagaatgagagttgcctaaattccagagagacctctttcataactaatgggagttgcct | |
| | ggcctccagaaagacctcttttatgatggccctgtgccttagtagtatttatgaagacttgaagatgtaccaggt | |
| | ggagttcaagaccatgaatgcaaagcttctgatggatcctaagaggcagatctttctagatcaaaacatgctg | |
| | gcagttattgatgagctgatgcaggccctgaatttcaacagtgagactgtgccacaaaaatcctcccttgaag | |
| | aaccggatttttataaaactaaaatcaagctctgcatacttcttcatgctttcagaattcgggcagtgactattgat | |
| | agagtgatgagctatctgaatgcttcctaa | |
| IRES | tccgcccctctccctaacgttactggccgaagccgcttggaataaggccggtgtgcgtttgtctatatgttatttt | SEQ ID |
| | ccaccatattgccgtcttttggcaatgtgagggcccggaaacctggccctgtcttcttgacgagcattcctagg | NO : 7 |
| | ggtctttcccctctcgccaaaggaatgcaaggtctgttgaatgtcgtgaaggaagcagttcctctggaagcttc | |
| | ttgaagacaaacaacgtctgtagcgaccctttgcaggcagcggaaccccccacctggcgacaggtgcctct | |
| | gcggccaaaagccacgtgtataagatacacctgcaaaggggcacaaccccagtgccacgttgtgagttg | |
| | gatagttgtggaaagagtcaaatggctctcctcaagcgtattcaacaaggggctgaaggatgcccagaagg | |
| | taccccattgtatgggatctgatctggggcctcggtgcacatgctttacatgtgtttagtcgaggttaaaaaaac | |
| | gtctaggccccccgaaccacggggacgtggttttcctttgaaaaacacgatgataatatggccacaacc | |
| ICP34.5 sgRNA | gttgcctgtcaaactctaccaccc | SEQ ID |
| sequence | | NO : 8 |
| UL26/27 sgRNA | caatgcaaaagcctttgacg | SEQ ID |
| sequence | | NO : 9 |
| UL3/4 sgRNA | caccgaataacacataaatttggc | SEQ ID |
| sequence | | NO : 10 |
| GFP sgRNA | ggcgagggcgatgccaccta | SEQ ID |
| sequence | | NO : 11 |
| hIL12 | atgtgtcaccagcagttggtcatctcttggttttccctggtttttctggcatctcccctcgtggccatatgggaact | SEQ ID |
| | gaagaaagatgtttatgtcgtagaattggattggtatccggatgcccctggagaaatggtggtcctcacctgtg | NO : 12 |
| | acacccctgaagaagatggtatcacctggaccttggaccagagcagtgaggtcttaggctctggcaaaacc | |
| | ctgaccatccaagtcaaagagtttggagatgctggccagtacacctgtcacaaaggaggcgaggttctaag | |
| | ccattcgctcctgctgcttcacaaaaaggaagatggaatttggtccactgatattttaaaggaccagaaagaa | |
| | cccaaaaataagacctttctaagatgcgaggccaagaattattctggacgtttcacctgctggtggctgacga | |
| | caatcagtactgatttgacattcagtgtcaaaagcagcagaggctcttctgacccccaaggggtgacgtgcg | |
| | gagctgctacactctctgcagagagagtcagaggggacaacaaggagtatgagtactcagtggagtgcca | |
| | ggaggacagtgcctgcccagctgctgaggagagtctgcccattgaggtcatggtggatgccgttcacaagc | |
| | tcaagtatgaaaactacaccagcagcttcttcatcagggacatcatcaaacctgacccacccaagaacttgca | |
| | gctgaagccattaaagaattctcggcaggtggaggtcagctgggagtaccctgacacctggagtactccac | |
| | attcctacttctccctgacattctgcgttcaggtccagggcaagagcaagagagaaaagaaagatagagtctt | |
| | cacggacaagacctcagccacggtcatctgccgcaaaaatgccagcattagcgtgcgggcccaggaccg | |
| | ctactatagctcatcttggagcgaatgggcatctgtgccctgcagttagctcgactccgcccctctccctaacg | |
| | ttactggccgaagccgcttggaataaggccggtgtgcgtttgtctatatgttattttccaccatattgccgtctttt | |
| | ggcaatgtgagggcccggaaacctggccctgtcttcttgacgagcattcctaggggtctttcccctctcgcca | |
| | aaggaatgcaaggtctgttgaatgtcgtgaaggaagcagttcctctggaagcttcttgaagacaaacaacgt | |
| | ctgtagcgaccctttgcaggcagcggaaccccccacctggcgacaggtgcctctgcggccaaaagccac | |
| | gtgtataagatacacctgcaaaggcggcacaaccccagtgccacgttgtgagttggatagttgtggaaaga | |
| | gtcaaatggctctcctcaagcgtattcaacaaggggctgaaggatgcccagaaggtaccccattgtatggga | |
| | tctgatctggggcctcggtgcacatgctttacatgtgtttagtcgaggttaaaaaaacgtctaggccccccgaa | |
| | ccacggggacgtggttttcctttgaaaaacacgatgataatatggccacaaccatgtggccccctgggtcag | |
| | cctcccagccaccgccctcacctgccgcggccacaggtctgcatccagcggctcgccctgtgtccctgcag | |
| | tgccggctcagcatgtgtccagcgcgcagcctcctccttgtggctaccctggtcctcctggaccacctcagtt | |
| | tggccagaaacctccccgtggccactccagacccaggaatgttcccatgccttcaccactcccaaaacctgc | |
| | tgagggccgtcagcaacatgctccagaaggccagacaaactctagaattttacccttgcacttctgaagagat | |
| | tgatcatgaagatatcacaaaagataaaaccagcacagtggaggcctgtttaccattggaattaaccaagaat | |
| | gagagttgcctaaattccagagagacctctttcataactaatgggagttgcctggcctccagaaagacctcttt | |
| | tatgatggccctgtgccttagtagtatttatgaagacttgaagatgtaccaggtggagttcaagaccatgaatg | |
| | caaagcttctgatggatcctaagaggcagatctttctagatcaaaacatgctggcagttattgatgagctgatg | |
| | caggccctgaatttcaacagtgagactgtgccacaaaaatcctcccttgaagaaccggatttttataaaactaa | |
| | aatcaagctctgcatacttcttcatgctttcagaattcgggcagtgactattgatagagtgatgagctatctgaat | |
| | gcttcctaa | |
| hPD-1 single | atgcaagtgcagctggtgcaaagcggcgtggaagtgaagaaacctggcgccagcgtgaaagtgtcctgca | SEQ ID |
| chain antibody | aggcctctggatatacatttaccaattactacatgtactgggtgcggcaggccccaggccagggcctggaat | NO : 13 |
| | ggatgggcggcatcaaccctagcaacggcggtacaaacttcaacgagaagttcaagaacagagtgaccct | |
| | gaccaccgacagcagcaccacaaccgcctacatggaactgaagagcctgcagttcgacgacaccgccgt | |
| | gtactactgcgccaggcgggactacagattcgacatgggctttgattactggggacagggcacaacagtca | |
| | ccgtgtccagcggcggcggaggctctggcggcggaggaagcggaggcggcggcagcgagatcgtgct | |
| | gacccagagccctgctacactctccctgagccccggcgagagagccaccctgagctgcagagccagcaa | |
| | gggcgtttctacatctggctacagctacctgcactggtaccagcagaagcccggccaggctcctagactgct | |
| | gatctacctggcctcttacctggagagcggcgtccccgctagattcagcggctctggcagcggaaccgattt | |
| | caccctgaccatcagctcccttgagcctgaggacttcgccgtgtactattgtcagcacagcagagatctgcct | |
| | ctgacattcggcggaggcaccaaggtggaaatcaagcggaccgtggcctag | |

### Example 2: Detection of Expression of Cytokine IL-12 After Infection of Cells With HSV1-hIL12(34.5 KO)-αhPD1(UL26/27) or HSV1-mIL12(34.5 KO)-αmPD1(UL26/27)

Vero cells were inoculated into 96-well plates at a density of 10000/well, and 50 μL of the original medium was aspirated the next day before infection. The virus was diluted to 1e+6 PFU/mL. Vero cells were infected with recombinant herpes simplex virus vectors HSV1-hIL12(34.5 KO) or HSV1-mIL12(34.5 KO) expressing the immunostimulatory molecule IL12 and the wild-type virus (wtHSV1) at multiplicity of infections MOI=3, respectively. After 2 hours of infection, the supernatant was aspirated, the cells were washed once with 100 μL of PBS, and the culture was continued after addition of 100 μL of DMEM medium. After 48 hours, the infected culture supernatant and cell lysate were harvested, respectively, and detection was performed using Human IL-12 p70 Quantikine HS ELISA Kit (R&D-HS120) or Mouse IL-12 p70 ELISA Kit (R&D-M1270) according to the Kit instructions. As shown in FIG. 3, it can be seen from the calculation of the standard curve that the concentration of IL-12 in the supernatant of the culture medium was higher than that in the cell lysate, wherein the concentration of IL12 in the supernatant of the culture medium was 2-5 ng/ml, and the concentration of IL12 in the cell lysate was about 1 ng/mL, while IL-12 was not expressed after infection with the wild-type virus.

Example 3: Proliferative Properties of HSV1-hIL12(34.5 KO)-αhPD1(UL26/27) in African Green Monkey Kidney Cells(Vero)

Virus species were grouped as follows:
1) HSV1-KOS(wt) is wild-type herpes simplex virus type 1;
2) HSV1-34.5 KO is a herpes simplex virus type 1 with ICP34.5 gene knocked out;
3) HSV1-hIL 12(34.5 KO) is a herpes simplex virus type 1 with the ICP34.5 gene replaced by hIL 12;
4) HSV1-hIL12(34.5 KO)-αhPD1(UL26/27) is a herpes simplex virus type 1 with the ICP34.gene replaced by hIL12 and the hPD-1 single chain antibody gene inserted between the viral genomes UL26/27.

Vero cells were inoculated into 96-well plates at a density of 10000/well. The next day, 4 different virus groups (HSV1-KOS(wt), HSV1-34.5 KO, HSV1-hIL12(34.5 KO), HSV1-hIL12(34.5 KO)-αhPD1(UL26/27)) were diluted with cell culture medium, respectively. 50 μL of the original culture medium was aspirated before infection, and the viruses were diluted to 1e+6 PFU/mL. Vero cells were infected at MOI=0.1, respectively. After 2 hours of infection, the supernatant was aspirated, the cells were washed once with 100 μL of PBS, and the culture was continued after addition of 100 μL of DMEM medium. Virus-infected culture supernatants and cell lysates were harvested at 24 hours, 48 hours, 72 hours and 96 hours after virus infection, respectively. Titers of the viruses harvested at different time points were determined on Vero cells as follows.

Vero cells were inoculated into 6-well plates at a density of 50 million/well. The next day, the harvested different viruses were diluted 10-fold with serum-free medium, and then 1 mL of virus at different dilution points was added to the 6-well plates. During 2-hour infection at 37° C., agarose was heated by an electromagnetic oven while a DMEM solution of 3.3% FBS was prepared, and both were placed in an incubator at 37° C., 5% CO2 for use. 5 minutes before infection was completed, 1 volume of 2.5% low melting point agarose solution was mixed with 1.5 volume of the DMEM solution of 3.3% FBS for use. After the infection, the infected liquid in the wells was discarded, 2 mL of the agarose mixed solution was added to each well, and then the plates were placed in a biological safety cabinet for 10-30 minutes to coagulate the agarose. Finally, the plates were placed in an incubator at 37° C., 5% CO₂ for another 72 hours to detect plaques. The virus titer at each time point was obtained by plaque statistics and the virus growth curve was plotted to determine the proliferative properties of the virus. As shown in FIG. 4, the recombinant virus HSV1-hIL12(34.5 KO)-αhPD1(UL26/27) has a proliferation capacity comparable to that of the wild-type virus.

### Example 4: Proliferative Properties of Different Constructs of the Same Viral Vector With Different Insertion Sites in Vero Cells

Grouping by insertion site is as follows:
A) HSV1-KOS(wt) is a wild-type herpes simplex virus type 1,
B) HSV1-hIL12(34.5 KO)-αhPD1(UL26/27) is a herpes simplex virus type 1 with the ICP34.5 gene replaced by hIL12 and the hPD-1 single chain antibody gene inserted between the viral genomes UL26/27,
C) HSV1-hIL12(34.5 KO)-αhPD1(UL3/4) is a herpes simplex virus type 1 with the ICP34.5 gene replaced by hIL12 and the hPD-1 single chain antibody gene inserted between the viral genomes UL3/4.

Vero cells were inoculated into 96-well plates at a density of 10000/well. The next day, 3 different viruses (HSV1-KOS(wt), HSV1-hIL12(34.5 KO)-αhPD1(UL26/27), HSV1-hIL12(34.5 KO)-αhPD1(UL3/4)) were diluted with cell culture medium, respectively. 50 μL of the original culture medium was aspirated before infection, and the viruses were diluted to 1e+6 PFU/mL. Vero cells were infected at MOI=0.1, respectively. After 2 hours of infection, the supernatant was aspirated, the cells were washed once with 100 μL of PBS, and the culture was continued after addition of 100 μL DMEM medium. Virus-infected culture supernatants and cell lysates were harvested at 24 hours, 48 hours, 72 hours, and 96 hours after virus infection. Titers of the harvested viruses were also determined on Vero cells.

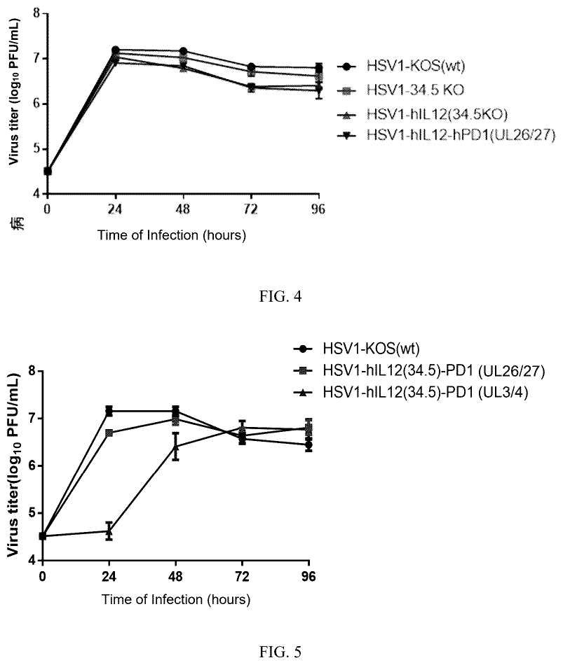

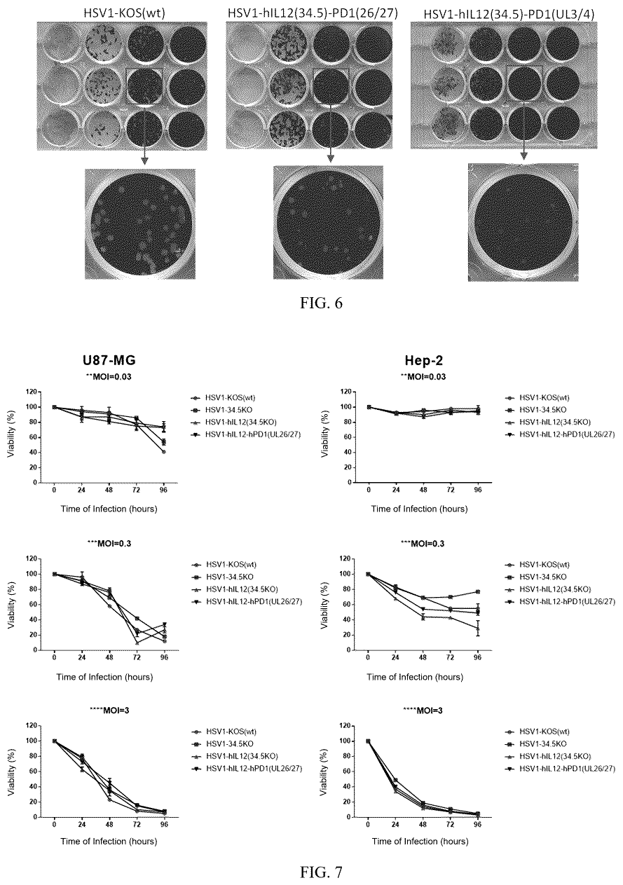

Vero cells were inoculated into 6-well plates at a density of 50 million/well. The next day, the harvested virus was diluted 10-fold with serum-free medium, and then 1 mL of virus at different dilution points was added to the 6-well plates. During 2-hour infection at 37° C., agarose was heated by an electromagnetic oven while a DMEM solution of 3.3% FBS was prepared, and both were placed in an incubator at 37° C., 5% CO₂ for use. 5 minutes before infection was completed, 1 volume of 2.5% low melting point agarose solution was mixed with 1.5 volume of the DMEM solution of 3.3% FBS for use. After the infection, the infected liquid in the wells was discarded, 2 mL of the agarose mixed solution was added to each well, and then the plates were placed in a biological safety cabinet for 10-30 minutes to coagulate the agarose. Finally, the plates were placed in an incubator at 37° C., 5% CO₂ for another 72 hours to detect plaques. The virus titer at each time point was obtained by plaque statistics and the virus growth curve was plotted to determine the proliferative properties of the virus. As shown in FIG. 5, the proliferation capacity of HSV1-hIL12(34.5 KO)-αhPD1(UL26/27) was significantly improved compared to HSV1-hIL12(34.5 KO)-αhPD1(UL3/4). FIG. 6 shows plaque assay results at 48 hours after viral infection, with hIL12(34.5 KO)-αhPD1(UL26/27) forming larger and more plaques than HSV1-hIL12(34.5 KO)-αhPD1(UL3/4) after infection.

### Example 5: In Vitro Oncolytic Activity of HSV1-hIL12(34.5 KO)-αhPD1(UL26/27) on Different Tumor Cells

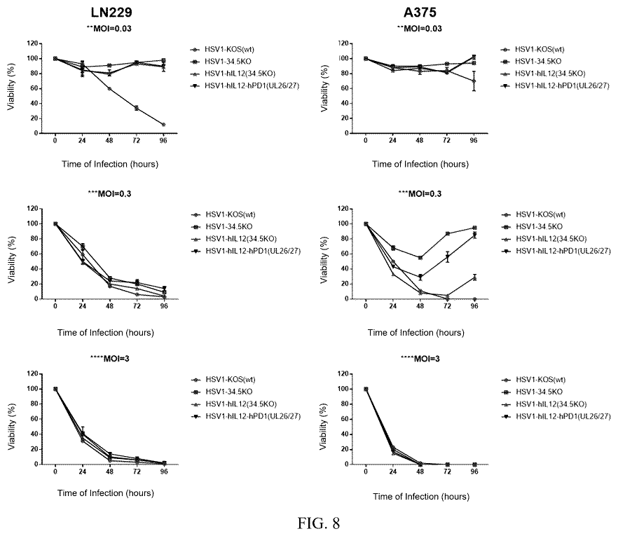

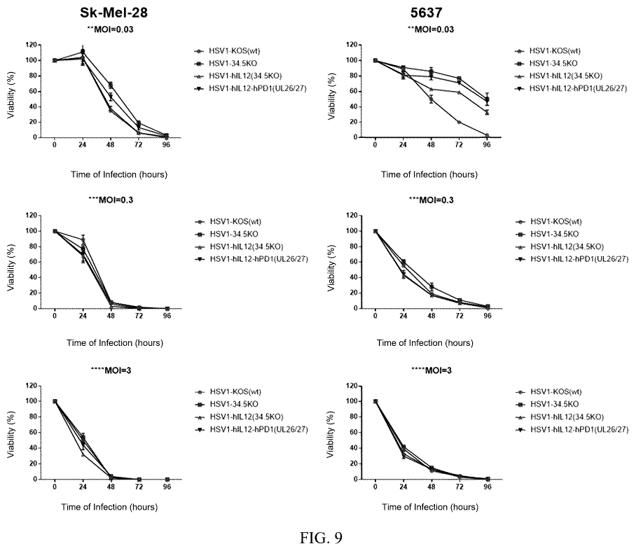

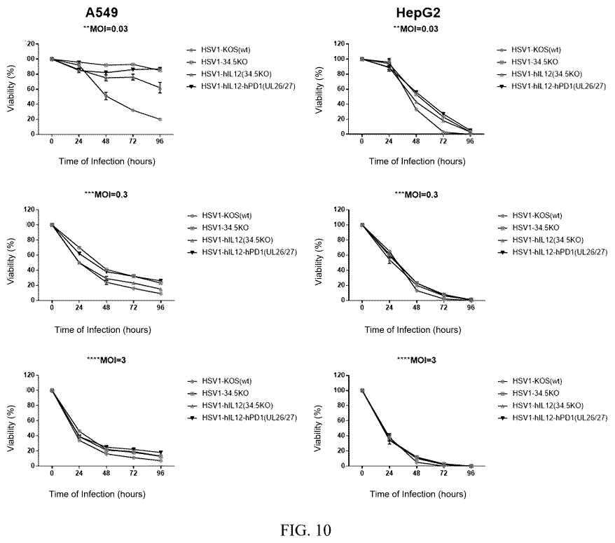

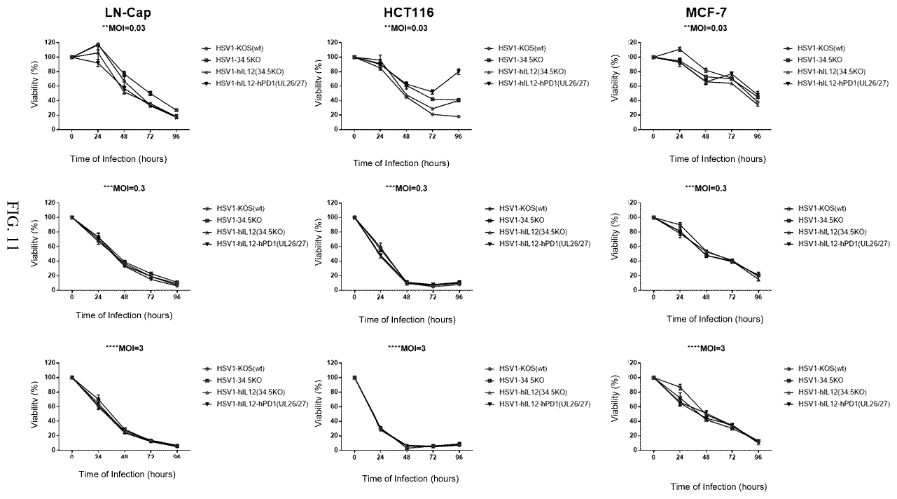

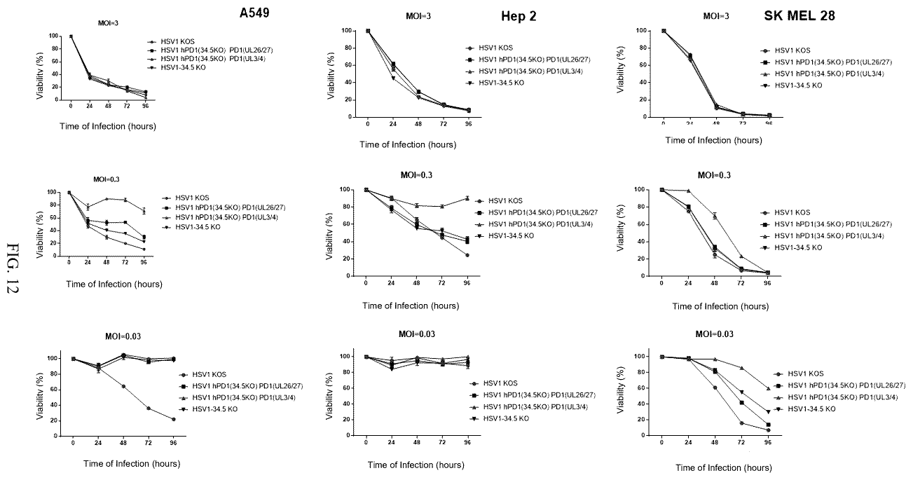

Eleven different tumor cells were inoculated into a 96-well plate. On the next day, 50 μL of the original medium was aspirated before infection, and the virus was diluted to 1e+6 PFU/mL. Different tumor cells were infected with the 4 different viruses in Example 3 at different multiplicity of infection MOI=0.03/0.3/3, respectively, and the specific cell types and culture information are shown in TABLE 4. After 2 hours of infection, the supernatant was aspirated. The cells were washed once with 100 μL of PBS, and the culture was continued after addition of 100 μL of DMEM medium. CCK-8 reagent was added every 24 hours and the incubation was terminated at 37° C. for 2 hours. The absorbance values of the culture broth were measured at 450 nm and 630 nm. The viabilities of the tumor cells infected by the virus were calculated by the read absorbance values of the culture solution. As shown in FIGS. 7-11, it can be seen from the cell viability plots of the infected cells at different time points that the virus HSV1-hIL12(34.5 KO)-αhPD1(UL26/27) can obviously kill various tumor cells, and when the multiplicity of infection is 0.03, the HSV1-hIL12(34.5 KO)-αhPD1(UL26/27) can completely kill the melanoma cells SK-MEL-28, hepatoma cells HepG2 and prostate cancer cells LN-Cap after 96 hours.

### Example 6: Comparison of HSV1-hIL12(34.5 KO)-αhPD1(UL26/27) With Other Vectors

HSV1-hIL12(34.5 KO)-αhPD1(UL26/27) was prepared according to Example 1, and the in vitro oncolytic activity was detected according to the experimental conditions of Example 5. The recombinant virus of the disclosure had two antitumor immune regulators knocked in to enhance the antitumor cell immunity of the body, showing a good killing effect in an in vitro killing experiment of various tumor cells. Compared with the existing herpes simplex oncolytic virus on the market, such as T-VEC or ΔG47 (Delytact), the recombinant virus of the disclosure has exogenous immunoregulation genes inserted into multiple sites on the premise of ensuring the safety (knockout of neurovirulence factor ICP34.5 gene). Many reported recombinant herpes simplex oncolytic viruses generally have exogenous genes inserted between UL3/4 and show a good tumor killing effect (Menotti, Laura, and Elisa Avitabile. “Herpes simplex virus oncolytic immunovirotherapy: the blossoming branch of multimodal therapy.” International Journal of Molecular Sciences 21.21 (2020): 8310. Kennedy, Edward M., et al. “Design of an interferon-resistant oncolytic HSV-1 incorporating redundant safety modalities for improved tolerability.” Molecular Therapy-Oncolytics 18 (2020): 476-490. Thomas S, Kuncheria L, Roulstone V, et al. Development of a new fusion-enhanced oncolytic immunotherapy platform based on herpes simplex virus type 1 [J]. Journal for immunotherapy of cancer, 2019, 7(1): 1-17. Abstract 1455: Design of ONCR-177 base vector, a next generation oncolytic herpes simplex virus type-1, optimized for robust oncolysis, transgene expression and tumor-selective replication). The recombinant virus of the disclosure creatively inserts an exogenous immunoregulation gene between UL26/27, and can obtain a better tumor killing effect.

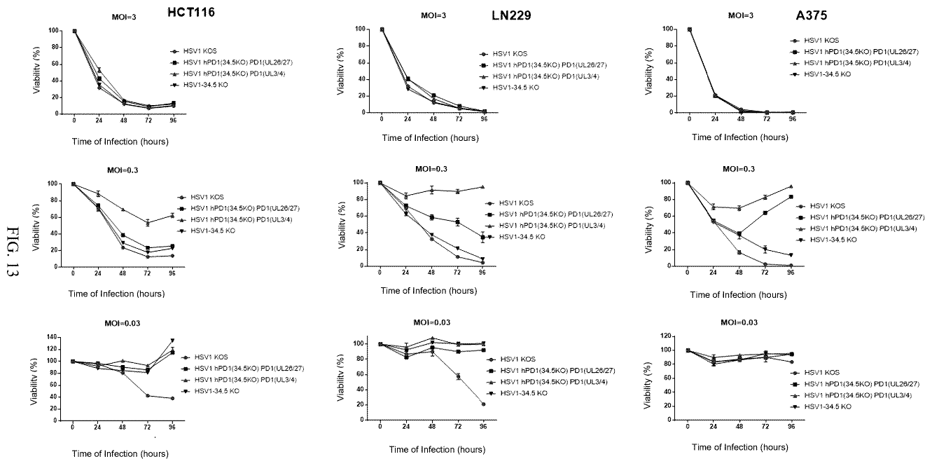

Therefore, the CRISPR/Cas9 technology is used for constructing recombinant HSV1 oncolytic virus in which an exogenous gene αhPD1 was inserted into the UL3/4 site, namely HSV1-hIL12(34.5 KO)-αhPD1(UL3/4), and HSV1-hIL12(34.5 KO)-αhPD1(UL3/4) was a herpes simplex virus with a 34.5 gene replaced by hIL12 and a hPD-1 single-chain antibody gene inserted between UL3/4 virus genomes. In contrast to HSV1-hIL12(34.5 KO)-αhPD1(UL26/27) prepared according to Example 1, it differs in the insertion site of hPD1, i.e., HSV1-hIL12(34.5 KO)-αhPD1(UL3/4) has hPD1 inserted between the genomic UL3/4. A variety of human tumor cells (A375, LN229, A549, SK-Mel-28, Hep2, HCT116) were selected and inoculated into 96-well plates at appropriate cell densities (see Table 4) and virus infection experiments were performed 18-24 hours later. Before infection, 50 μL of original culture medium was discarded, recombinant viruses of HSV1-hIL12(34.5 KO)-ahPD1(UL3/4), HSV1-hIL12(34.5 KO)-ahPD1(UL26/27) and the like were diluted to 1e+06 PFU/mL, and different tumor cells were infected at MOI×0.03/0.3/3, respectively. After 2 hours of infection, the supernatant was aspirated, the cells were washed once with 100 μL of PBS, and the culture was continued after addition of 100 μL of DMEM medium. After 24, 48, 72 and 96 hours of culture, CCK-8 reagent was added and the plate was incubated at 37° C. for 2 hours and then terminated. The absorbances of the culture were measured at 450 nm and 630 nm. The viabilities of the tumor cells infected by the virus were calculated through the read absorbance value of the culture solution. Cell viability plots of the infected cells at different time points were plotted to see the killing activity of the virus on corresponding tumor cells, and the killing activities of the constructed multiple oncolytic viruses on different tumor cells were compared. As shown in FIGS. 12-13, the recombinant viruses of the present disclosure showed better oncolytic killing effect compared to HSV1-hIL12(34.5KO)-αhPD1(UL3/4) in in vitro killing experiments of multiple tumor cells.

TABLE 4
Cell Culture Information

| | | | Viral |
|---|---|---|---|
| | | Cell | Infection |
| Cells | Medium Used | Density | Titer |
| A375 | DMEM + 10% FBS + 1% two | 10000/ | 0.03moi, |
| | antibiotics | well | 0.3moi, |
| LN229 | DMEM + 10% FBS + 1% two | | 3moi |
| | antibiotics | | |
| A549 | 1640 + 10% FBS + 1% two antibiotics | | |
| U87-MG | DMEM + 10% FBS + 1% two | | |
| | antibiotics | | |
| HepG2 | 1640 + 10% FBS + 1% two antibiotics | | |
| SK-Mel- | DMEM + 10% FBS + 1% two | | |
| 28 | antibiotics | | |
| Hep-2 | DMEM + 10% FBS + 1% two | | |
| | antibiotics | | |
| 5637 | 1640 + 10% FBS + 1% two antibiotics | | |
| LN-Cap | 1640 + 10% FBS + 1% two antibiotics | | |
| HCT116 | 1640 + 10% FBS + 1% two antibiotics | | |
| MEF-7 | DMEM + 10% FBS + 1% two | | |
| | antibiotics | | |

### Example 7: Evaluation of the In Vivo Oncolytic Activity of HSV1-hIL12(34.5 KO)-αhPD1(UL26/27)

Tumor cells SK-Mel-28 were cultured in an incubator at 37° C., 5% CO₂ using complete cell culture medium (see Table 4). When the cells reached the amount required for the construction of the mouse tumor model, the cells were digested with 0.05% trypsin and washed with PBS, and finally a cell suspension containing 50% matrigel (cell density 8e+7/mL) was obtained.

0.1 mL of SK-Mel-28 cell suspension was taken and inoculated subcutaneously on the backs of nude mice with the age of 4-6 weeks, respectively, for carrying out subcutaneous tumor modeling of human melanoma cell strain. When the tumor volume on the backs of the mice were about 100 mm³, the mice were grouped and treatment was initiated, with the treatment regimen as in TABLE 5 below.

Tumor volumes were measured 2 times per week and tumor size was measured with an electronic vernier caliper. Tumor volumes were calculated according to the formula: V (volume)=(L×(W)²)/2; L represents the tumor length and W represents the tumor width.

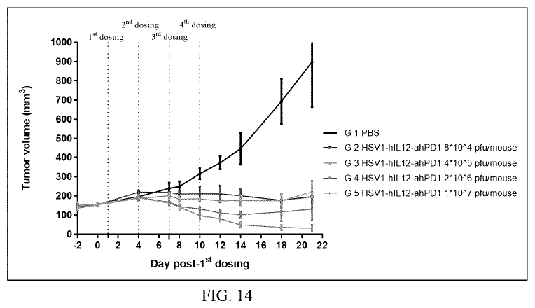

As shown in FIG. 14, the recombinant virus of the present disclosure still has an obvious tumor inhibition effect in a low-dose 8e+4 PFU/mouse and shows a good oncolytic killing effect in the in vivo model experiment of human melanoma cell SK-Mel-28.

TABLE 5
Treatment Regimen

| | | | Treatment Regimen | | |
|---|---|---|---|---|---|
| | | Animal | | Dosing | Fre- |
| Group # | | Numbers | Dosage | Route | quency |
| 1 | PBS | 6 | 100 μL | intratumor | Days 1, |
| 2 | HSV1-hIL12-ahPD1 | 8 | 1E+07 PFU/ | | 4, 7, 10 |
| 3 | HSV1-hIL12-ahPD1 | 8 | mouse | | |
| 4 | HSV1-hIL12-ahPD1 | 8 | 2E+06 PFU/ | | |
| | | | mouse | | |
| | | | 4E+05 PFU/ | | |
| | | | mouse | | |
| 5 | HSV1-hIL12-ahPD1 | 8 | 8E+04PFU/ | | |
| | | | mouse | | |

### Example 8: Recombinant Herpes Simplex Virus Vector for Expressing Immunostimulation Factor and Biological Function Thereof

On the basis of HSV1-KOS strain (ATCC-VR-1544, GHSV-UL46), with reference to the method of Example 1, gene editing technology CRISPR/Cas9 was used to knock out neurovirulence protein ICP34.5 (SEQ ID NO: 1). A two-step method was used to delete the ICP34.5 gene and replace it with an IL-2 sequence (gene sequence Genbank Accession No.: NM_000586.4; amino acid sequence Genbank Accession No.: NP_000577.2), and a CTLA-4 gene (gene sequence Accession No.: NM_001037631.3; amino acid sequence Genbank Accession No.: NP_001032720.1) was inserted between virus genomic UL26/27. Thereby, a recombinant herpes simplex virus vector for expressing immunostimulatory factor was constructed.

On the basis of HSV1-KOS strain (ATCC-VR-1544, GHSV-UL46), with reference to the method of Example 1, gene editing technology CRISPR/Cas9 was used to knock out neurovirulence protein ICP34.5 (SEQ ID NO: 1). A two-step method was used to delete the ICP34.5 gene and replace it with an IL-2 sequence (gene sequence Genbank Accession No.: NM_000586.4; amino acid sequence Genbank Accession No.: NP_000577.2), and an anti-PD-L1 antibody gene (VH amino acid sequence DrugBank Accession Number: DB11595; VL amino acid sequence DrugBank Accession Number: DB11595) was inserted between virus genomic UL26/27. Thereby, a recombinant herpes simplex virus vector for expressing an immunostimulatory factor was constructed.

On the basis of HSV1-KOS strain (ATCC-VR-1544, GHSV-UL46), with reference to the method of Example 1, gene editing technology CRISPR/Cas9 was used to knock out neurovirulence protein ICP34.5 (SEQ ID NO: 1). A two-step method was used to delete the ICP34.5 gene and replace it with a GM-CSF sequence (gene sequence Genbank Accession No.: NM_000758.4; amino acid sequence Genbank Accession No.: NP_000749.2), and a CTLA-4 gene (gene sequence Accession No.: NM_001037631.3; amino acid sequence Genbank Accession No.: NP_001032720.1) was inserted between virus genomic UL26/27. Thereby, a recombinant herpes simplex virus vector for expressing an immunostimulatory factor was constructed.

On the basis of HSV1-KOS strain (ATCC-VR-1544, GHSV-UL46), with reference to the method of Example 1, gene editing technology CRISPR/Cas9 was used to knock out neurovirulence protein ICP34.5 (SEQ ID NO: 1). A two-step method was used to delete the ICP34.5 gene and replace it with a GM-CSF sequence (gene sequence Genbank Accession No.: NM_000758.4; amino acid sequence Genbank Accession No.: NP_000749.2), and an anti-PD-L1 antibody gene (VH amino acid sequence DrugBank Accession Number: DB11595; VL amino acid sequence DrugBank Accession Number: DB11595) was inserted between virus genomic UL26/27. Thereby, a recombinant herpes simplex virus vector for expressing an immunostimulatory factor was constructed.

On the basis of HSV1-KOS strain (ATCC-VR-1544, GHSV-UL46), with reference to the method of Example 1, gene editing technology CRISPR/Cas9 was used to knock out neurovirulence protein ICP34.5 (SEQ ID NO: 1). A two-step method was used to delete the ICP34.5 gene and replace it with an IL-15 sequence (gene sequence Genbank Accession No.: NR_037840.3; amino acid sequence Genbank Accession No.: NP_000576.1), and a CTLA-4 gene (gene sequence Accession No.: NM_001037631.3; amino acid sequence Genbank Accession No.: NP_001032720.1) was inserted between virus genomic UL26/27. Thereby, a recombinant herpes simplex virus vector for expressing an immunostimulatory factor was constructed.

On the basis of HSV1-KOS strain (ATCC-VR-1544, GHSV-UL46), with reference to the method of Example 1, gene editing technology CRISPR/Cas9 was used to knock out neurovirulence protein ICP34.5 (SEQ ID NO: 1). A two-step method was used to delete the ICP34.5 gene and replace it with an IL-15 sequence (gene sequence Genbank Accession No.: NR_037840.3; amino acid sequence Genbank Accession No.: NP_000576.1), and an anti-PD-L1 antibody gene (VH amino acid sequence DrugBank Accession Number: DB11595; VL amino acid sequence DrugBank Accession Number: DB11595) was inserted between virus genomic UL26/27. Thereby, a recombinant herpes simplex virus vector for expressing an immunostimulatory factor was constructed.

The different constructs obtained were evaluated with respect to cytokine expression after infection of the cells, proliferation profile of infection of Vero cells and oncolytic activity in vitro and in vivo on different tumor cells, with reference to the experimental methods for biological activity described in Examples 2-7. The results show that the virus vectors have better oncolytic killing effect.

1. A recombinant HSV-1 vector comprising a modified HSV-1 genome, wherein the modification comprises a deletion of the ICP34.5 gene in a HSV1-KOS strain genome, and the recombinant HSV-1 vector comprises: (a) a first exogenous nucleic acid sequence encoding an immunostimulatory factor; and (b) a second exogenous nucleic acid sequence encoding an anti-immune checkpoint antibody; wherein an insertion site of the first exogenous nucleic acid sequence or the second exogenous nucleic acid sequence is between the UL26 and UL27 genes of the HSV1-KOS strain genome, and the insertion of the first exogenous nucleic acid sequence or the second exogenous nucleic acid sequence does not interfere with an expression of HSV-1.

2. The recombinant HSV-1 vector of claim 1, wherein the insertion site of the first exogenous nucleic acid sequence encoding an immunostimulatory factor is in a region of the deleted ICP34.5 gene in the HSV1-KOS strain genome or between the UL3 and UL4 genes in the HSV1-KOS strain genome, and the insertion site of the second exogenous nucleic acid sequence encoding the anti-immune checkpoint antibody is between the UL26 and UL27 genes in the genome of the HSV1-KOS strain; or the insertion site of the first exogenous nucleic acid sequence encoding the immunostimulatory factor is between the UL26 and UL27 genes of the HSV1-KOS strain genome, and the insertion site of the second exogenous nucleic acid sequence encoding the anti-immune checkpoint antibody is in a region of the deleted ICP34.5 gene in the HSV1-KOS strain genome or between the UL3 and UL4 genes of the HSV1-KOS strain genome.

3. The recombinant HSV-1 vector of claim 1, wherein the immunostimulatory factor is at least one selected from GM-CSF, IL-2, IL-12, IL-15, IL-24, and IL-27; and the immune checkpoint is at least one selected from PD-1, CTLA-4, VISTA, LAG-3, TIGIT, and PD-L1.

4. The recombinant HSV-1 vector of claim 1, wherein the HSV-1 vector comprises: (a) a first exogenous nucleic acid sequence encoding IL-12; (b) a second exogenous nucleic acid sequence encoding an anti-PD-1 antibody.

5. The recombinant HSV-1 vector of claim 1, wherein the deletion of the ICP34.5 gene is a two-copy deletion of the ICP34.5 gene or a deletion of amino acid positions 1-146 of the N-terminal sequence of the ICP34.5 gene.

6. The recombinant HSV-1 vector of claim 1, wherein the nucleic acid sequence of the ICP34.5 gene comprises the sequence shown in SEQ ID NO:1.

7. The recombinant HSV-1 vector of claim 1, wherein the immunostimulatory factor or anti-immune checkpoint antibody is selected from the group consisting of murine, human, primate, and chimeric factors or antibodies.

8. The recombinant HSV-1 vector of claim 1, wherein the anti-immune checkpoint antibody is an intact antibody, a single-chain antibody or an antibody fragment.

9. The recombinant HSV-1 vector of claim 3, wherein the nucleic acid sequence of the anti-PD-1 antibody comprises the sequence shown in SEQ ID NO:2.

10. The recombinant HSV-1 vector of claim 3, wherein the first exogenous nucleic acid sequence encoding IL-12 comprises a sequence obtained by tandemly linking SEQ ID NO:3 and SEQ ID NO:4 via an IRES.

11. The recombinant HSV-1 vector of claim 1, wherein the HSV-1 vector further comprises a promoter sequence operably linked to the first exogenous nucleic acid sequence or the second exogenous nucleic acid sequence.

12. The recombinant HSV-1 vector of claim 11, wherein the promoter is at least one selected from CMV, SV40, EF1A, CBh and CAG promoters.

13. The recombinant HSV-1 vector claim 1, wherein the deletion of the ICP34.5 gene is a two-copy deletion of the ICP34.5 gene, and wherein the insertion of the first exogenous nucleic acid sequence encoding the immunostimulatory factor is a two-copy insertion or a single-copy insertion, and the insertion of the second exogenous nucleic acid sequence encoding the anti-immune checkpoint antibody is a single-copy insertion.

14. A pharmaceutical composition or a kit, comprising a therapeutically effective amount of the recombinant HSV-1 vector of claim 1, and one or more pharmaceutically acceptable carriers, diluents, buffers, or excipients.

15. A method for treating and/or preventing a cancer, comprising administering the recombinant HSV-1 vector of claim 1 to a subject.

16. The use method of claim 15, wherein the cancer is at least one selected from a gallbladder cancer, bladder cancer, basal cell tumor, extrahepatic cholangiocarcinoma, colorectal cancer, endometrial cancer, cervical cancer, esophageal cancer, breast cancer, Ewing's sarcoma, prostate cancer, gastric cancer, glioma, Hodgkin's lymphoma, laryngeal cancer, liver cancer, lung cancer, melanoma, mesothelioma, pancreatic cancer, renal cancer, peripheral nerve tumor, skin and plexiform neurofibroma, leiomyomatoid tumor, fibroma, uterine fibroids, leiomyosarcoma, thyroid cancer, ascites, mesothelioma, salivary gland tumor, mucoepidermoid carcinoma of the salivary gland, acinar cell carcinoma of the salivary gland, gastrointestinal stromal tumor (GIST), tumors causing accumulation of fluid in potential spaces of the body, pleural effusion, pericardial effusion, peritoneal effusion, giant cell tumor, pigmented villonodular synovitis (PVNS), tenosynovial giant cell tumor (TGCT), and sarcoma.

17. The method of claim 15, wherein the administration of the medicament is local administration or systemic administration, wherein the local administration includes intratumoral injection, and the systemic administration includes oral administration and intravascular injection.

18. The method of claim 16, wherein the liver cancer is hepatocellular carcinoma (HCC), cholangiocellular carcinoma or mixed type of liver cancer; the lung cancer is non-small cell cancer or small cell lung cancer; the peripheral nerve tumor is a malignant peripheral nerve sheath tumor (MPNST); the thyroid cancer is at least one selected from papillary thyroid cancer, anaplastic thyroid cancer, medullary thyroid cancer, follicular thyroid cancer, and Hurthle cell carcinoma; the ascites is malignant ascites; the giant cell tumor is a giant cell tumor of bone or a giant cell tumor of the tendon sheath; the salivary gland tumor is mucoepidermoid carcinoma of the salivary gland or acinar cell carcinoma of the salivary gland; the laryngeal cancer is laryngeal mucoepidermoid carcinoma; and/or the colorectal cancer is colon cancer or rectal cancer.

19. The recombinant HSV-1 vector of claim 7, wherein the immunostimulatory factor or anti-immune checkpoint antibody is selected from human factors or antibodies.

20. The recombinant HSV-1 vector of claim 10, wherein the IRES sequence comprises a sequence shown in SEQ ID NO:5.
